(19) United States
(12) Patent Application Publication
(10) Pub. No.: US 2023/0408391 A1
(43) Pub. Date: Dec. 21, 2023

(54) CHEWING MACHINE

(71) Applicant: UNIVERSIDAD TÉCNICA FEDERICO SANTA MARÍA, Valparaíso (CL)

(72) Inventors: Cristian Alejandro RAMIREZ BUSTOS, Viña del Mar (CL); Rolando Rafael ESPARZA DELGADO, Valparaíso (CL); Ricardo José SIMPSON RIVERA, Viña del Mar (CL); Rosendo Francisco ALAMOS TAPIA, Viña del Mar (CL)

(21) Appl. No.: 18/037,500

(22) PCT Filed: Nov. 15, 2021
(86) PCT No.: PCT/CL2021/050110

§ 371 Date: May 17, 2023

(30) Foreign Application Priority Data
Nov. 17, 2020 (CL) 2985-2020

(51) Int. Cl. G01N 3/38; G01N 33/02

(52) U.S. Cl. CPC G01N 3/38; G01N 33/02; G01N 2203/0007; G01N 2203/0025; G01N 2203/0087; G01N 2203/0019

(57) ABSTRACT

The present invention relates to an in vitro system for automatic chewing of solid and semi-solid food intended to simulate the human chewing process of a food sample, comprising a lower toothed ring and an upper toothed ring; and further comprising a force lever consisting of an arm from which a lead weight hangs, which is moved along the arm via the traction exerted by an electric motor controlled by a microcontroller board, which is connected by a spindle to the upper toothed ring; and wherein the shearing action is provided by an electric motor controlled by a microprocessor board which is connected to the shaft of the upper toothed ring via a chain. A second objective of the invention comprises a method for automatic chewing with controlled force and a specific number of chewing cycles and shear angle.

### FIELD OF APPLICATION

The present invention relates to an in vitro system and method for automatic mastication of solid and semi-solid foods intended to simulate the human chewing process of a food sample. More specifically to a mechanical system which by applying a specified force, a specified number of compression cycles, and a specified shear angle automatically crushes food by generating a particle size distribution.

### DESCRIPTION OF PRIOR ART

An in vitro automatic chewing system is a structure intended to reduce the size of solid and semi-solid foods by continuous, force-controlled compressions, and is properly composed of three complementary devices: the sample container, the serrated compression rings, and the support apparatus. In general, chewing tests associated with size reduction of solid and semi-solid foods are performed directly with people, and there is no system on the market that simulates the chewing process.

In an in vitro automatic chewing system, once the solid or semi-solid food is chewed, the size distribution produced can vary according to the force applied in the chewing process, as well as the number of times the solid or semi-solid food is chewed and the shear angle.

In general, continuous, shear, and force-controlled compression is intended to produce a particle size distribution of solid and semi-solid foods that allows to establish quantitative relationships between chewing conditions and bolus formation and nutrient release in in vitro digestion studies. The use of the automatic chewing system to obtain size distributions of solid and semi-solid foods is referred to laboratory experiments, in which a force lever is used, which pushes the jaws against each other allowing compression and an electric motor that allows the proportional rotation of the jaws allow the shear between them. It is only suitable for solid and semi-solid foods.

U.S. Pat. No. 7,721,456 (B2) dated May 25, 2010, by MARICHI RODRIGUEZ Francisco Javier et al, entitled “Measuring Apparatus For The Programming And Welding Of Adjustable Brackets” by Marichi, describes a measuring apparatus for the programming and welding of adjustable brackets permitting the electrical spot welding with a great accuracy of the elements of an adjustable bracket, in this manner being able to adjust or to program the information that the orthodontist wants to incorporate in the bracket in regard to inclination, angulation and rotation. In order to achieve this, he developed two attachments: the position measuring accessory and the welding accessory. The position measuring accessory has the function of recording or measuring accurately the dental position as for inclination, angulation and rotation on dental casts. This measurement is performed by means of a template system that allows the recording a tooth position in relation to its osseous basis and the occlusal plane, the invention has the innovation of allowing the accurate welding of the adjustable bracket elements (base and body), being able to adjust or program (in the three space planes) the information of inclination, angulation and rotation that the orthodontist wants to incorporate in the bracket by means of three fine graduation systems.

Patent application CN107260188 (A) dated Oct. 20, 2017, by Lu Xi et al. entitled “Multidirection biting force measuring device based on force sensor” describes a multidirectional biting force measuring device based on a force sensor. The multidirectional biting force measuring device comprises a removable outer cover and a plurality of groups of measuring systems arranged on the outer cover, wherein the outer cover comprises an upper cover body and a lower cover body, the plurality of groups of measuring systems comprises a main measuring system with one end connected to the inner wall of the outer casing and the other end pushing against the inner wall of the outer casing, and an auxiliary measuring system with one end connected to the main measuring system and the other end connected to the inner wall of the upper casing body or the inner wall of the lower casing body. The multidirectional biting force measuring device based on the force sensor overcomes the defect that an existing instrument cannot measure the specific direction of the bite force, the real-time positive pressure is measured based on the pressure sensors of the four-way measurement systems, the real-time measurement of the magnitude and direction of the bite force when a pair of upper and lower teeth chew an object, the multi-directional bite force measurement device can be used for the diagnosis and treatment of oral diseases, the bite force measurement device provided by the invention can be used repeatedly, and the spherical outer shell can also provide a true chewing sensation when measuring a person's bite force.

Patent U.S. Pat. No. 5,357,973 (A) dated 10.25.1994, by Sunouchi Yujiro et al, entitled “Measuring system for vital muscle activity” describes a measuring device that can easily measure and analyze the activity of the muscles of the living organism, for instance, measure the strength of occlusion of the masticatory muscles and duration thereof. The vital muscle activity measurement device comprises an amplifier means brought in contact with the muscles of a subject for detecting and amplifying a muscle current, an envelope forming means for forming an envelope waveform of the output of the amplifier means, and a timer means for determining the time for which the level of the envelope waveform obtained by the envelope forming means exceeds a preset reference level.

Patent U.S. Pat. No. 3,708,882 (A) dated 1 Sep. 1973, by Guichet N, entitled “Dental articulator accessory” describes dental supports for clutches in an articulator are described which are used to facilitate the mounting of the clutches and their dependent dental instruments in an articulator. The supports comprise a base member which is removably attached to the dental cast support screw of each articulator frame member with a telescoping member that bears a clutch support plate and that can be locked to the base member at any desired extension therein. The supports are used when the adjustable fossa and incisal guides of the articulator are to be set to duplicate a patient's border mandibular movements. In this method, a pantograph with attached clutches is positioned in the articulator in the proper anatomical relationship to the articulator control surfaces. The clutch support plates of the accessory of this invention are then extended from their base members into proximate positions to their respective clutches, a curable plastic is placed between the clutches and the support plates, and the articulator is maintained in its centric position until the plastic cements the clutches to their support plates.

Patent application US2008261169 (A1) dated Oct. 23, 2008, by Gutman Yevsey et al. entitled “Apparatus and method for replicating mandibular movement” describes an apparatus to replicate and analyze movement of a mandible relative to a maxilla with dental casts thereof includes a base frame, an arm connected to the base frame and a suspension assembly positionable by a plurality of electro-mechanical actuators. The arm connects to the base frame and supports the dental cast of the maxilla in a fixed position relative to the base frame. The suspension assembly, having opposing first and second sides, supports the dental cast of the mandible in working relation to the dental cast of the maxilla. The plurality of actuators each selectively impart movement to the sides of the suspension assembly such that the movement of the mandible obtained during a recording process can be replicated on the apparatus in real time.

None of the cited documents describe an in vitro automatic chewing system and method for solid and semi-solid food intended to simulate the human chewing process of a food sample.

### SUMMARY OF THE INVENTION

A first subject matter of the invention describes an in vitro automatic chewing system, comprising: a sample container which is formed by the lower toothed ring which is mounted on a circular aluminum base and enclosed by a glass cylinder. In the center of the lower ring there is a conical Teflon assembly protruding above the lower ring. The sample container is mounted on the lower shaft of the unit, which is connected by a chain pulley to a motor that allows the Y-axis movement of the sample container and operates according to the specified number of chewing cycles. The upper ring is mounted on a Teflon base, which has a conical space in the center that coincides with the lower ring. The upper ring has a clamping system to the upper shaft which connects to the weights controlling the compression force, through a lead weight whose mass moves along a force lever by means of a spindle system which is controlled by an electric motor. Based on the lever principle, the force will vary according to the distance of the weight with respect to the pivot point. The opposite end of the lever is connected to the shaft supporting the upper ring. Shearing is achieved by a motor coupled to the shaft of the upper ring by a pair of sliding gears coupled by a chain, which allows the upper ring to rotate around its axis on the lower ring at a specific angle.

A second subject matter of the invention comprises a method for automatic chewing with controlled force and specific number of chewing cycles and shear angle, comprising having an in vitro automatic chewing system, in accordance with the first subject matter of the invention; which includes the steps of:
Pressing the power switch of the in vitro automatic chewing system,
Setting the force parameters, number of chewing cycles, and shear angle,
Placing the solid or semi-solid food sample homogeneously distributed in the sample container,
Initiating the chewing process, and
Once the chewing process is finished, removing the crushed food from the sample container.

### BRIEF DESCRIPTION OF THE FIGURES

FIG. 1 depicts a main isometric view of the in vitro automatic chewing system of the invention.

FIG. 2 depicts another main isometric view of the in vitro automatic chewing system with touch control keypad and indicator display of the invention.

FIG. 3 depicts side views of the in vitro automatic chewing system of the invention.

FIG. 4 depicts a front view of the in vitro automatic chewing system of the invention.

FIG. 5 depicts a front view of the interior of the in vitro automatic chewing system of the invention.

FIG. 6 depicts a rear view of the interior of the in vitro automatic chewing system of the invention.

FIG. 7A depicts a detailed bottom view of the in vitro automatic chewing system of the invention.

FIG. 7B depicts a top view of the in vitro automatic chewing system of the invention.

### DESCRIPTION OF A PREFERRED EMBODIMENT

The first objective of the invention is to provide an in vitro automatic chewing system, which crushes a food with programmable force parameters, number of chewing cycles, and shear angle according to the user's previous requirements.

The force lever is the main physical component of the in vitro automatic chewing system proposed herein. The configuration of the force to be applied during mastication is achieved based on the lever principle, the force will vary according to the distance of the weight with respect to the pivot point through a lead weight of known mass and that according to the selected force moves along an inclined arm or force lever through a spindle system that is controlled with an electronically controlled electric motor to rotate in both directions, which can be a servomotor, stepper motor, or similar with the required force according to specifications. The opposite end of the lever is connected to the shaft supporting an upper toothed ring by means of a cord, this ring is responsible for transferring the force projected from the force lever to the solid or semi-solid food.

A connecting rod mechanism, which is coupled by a chain to an electronically controlled electric motor to rotate in both directions, which can be a servomotor, stepper motor, or similar with the required force according to specifications, allows the vertical movement of a lower toothed ring.

A motor coupled to the shaft of the upper toothed ring by means of a pair of sliding gears coupled by a chain allows the upper ring to rotate around its axis on the lower ring at specific angles, obtaining the shear between the two toothed rings.

The two toothed rings, upper and lower, are each formed by 21 molars made of resin and designed from the mold of normal human teeth, between which the solid or semi-solid food is located. The lower tooth ring is mounted on an aluminum plate and protected by a cylindrical cup.

The parameter setting, control and automation of the chewing process is handled by an electronic controller board, Arduino ATMega 2560 type, which has a series of digital and analog inputs/outputs, which are monitored and controlled using a programming language previously designed specifically for the functions enabled in the equipment. The electronic controller board is also in charge of monitoring a series of digital control and protection switches located in the different mechanisms of the equipment. The reading and setting of the different control parameters are entered by the user by means of a universal membrane type keyboard and are visualized by means of a 2×13 LCD screen, which are also read and controlled by the electronic controller card.

According to the mentioned mechanism, a first half of a chewing cycle starts when the lower toothed ring rises and impacts with the upper toothed ring, both toothed rings rise together to a specific distance, driven by the force of the lower electric motor, at which time the force is applied on the solid or semi-solid food. At the same time, the motor connected to the shaft of the upper toothed ring produces the circular motion at a specific angle, obtaining the shear between both toothed rings. Once both toothed rings are raised to their highest position, the second half of the chewing cycle begins, when the lower disc descends to its lowest position to start a new chewing cycle.

### Detailed Description of the Chewing System:

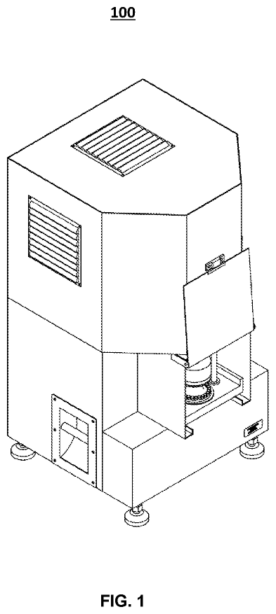

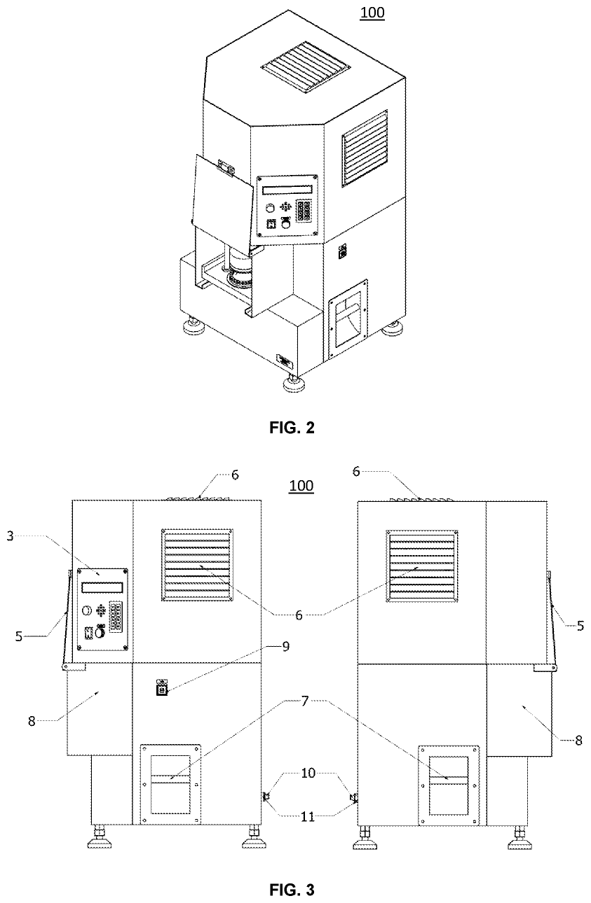

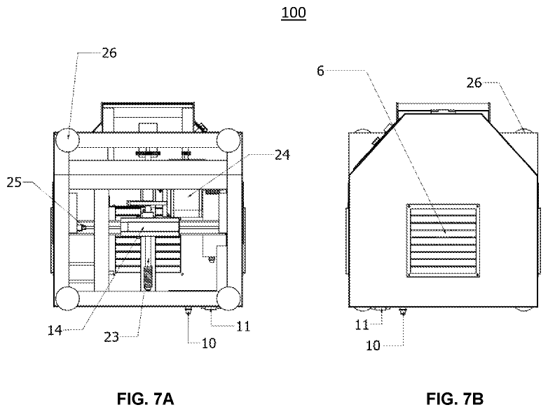

The in vitro automatic chewing system (100) is shown in FIGS. 1 to 7. FIGS. 1, 2, 3, and 4 describe the external components that allow the manipulation of the in vitro automatic chewing system (100) formed by a control panel (3), a feeding chamber (8) where the sample to be masticated and its safety cover (5) are located, ventilation grids (6), transport handles (7), lighting system (4), USB connector for data transfer (9), fuse (10), 220V power supply connector (11), and leveling feet (26).

FIG. 1 depicts the main component for reading and setting the different control parameters, which are entered by the user through a universal membrane type keyboard and are visualized by means of a 2×13 LCD display (3), which are also read and controlled by the microcontroller board.

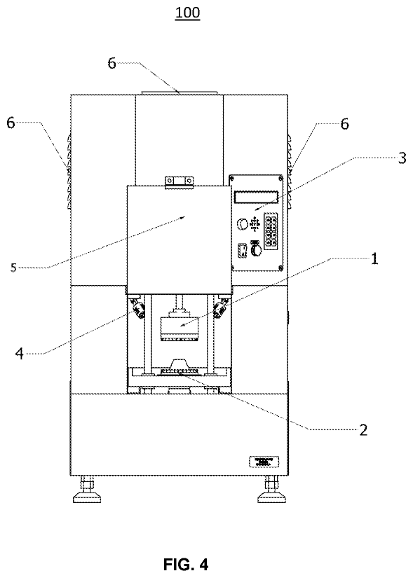

FIG. 3 depicts the main components that allow the mastication of solid and semi-solid food inside the feeding chamber (8) according to the parameters set with the control panel (3). An upper toothed ring (1) and a lower toothed ring (2), shown in FIG. 4, are each formed by 21 molars made of resin and designed from the mold of normal human teeth, between which the solid or semi-solid food is located.

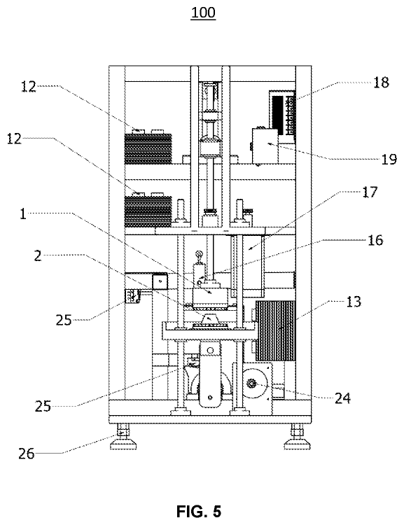

FIG. 5 depicts the main components that allow the vertical movement of the lower toothed ring (2) and exert the pressure on the upper toothed ring (1) to produce the breakage of the food. A connecting rod mechanism (25), which is coupled by a chain to a first lower electric motor (24) and electronically controlled with a first microstep driver controller (CW-8060) (13), that allows rotating in both directions and allows the vertical movement of the lower toothed ring (2); furthermore, the main components that allow a shearing movement of the upper toothed ring (1) are shown. A second electric motor (17) powered by an electric current source (20) and electronically controlled by a second microstep driver controller (CW-5045) (12) coupled to the shaft of the upper toothed ring (1) by means of a pair of sliding gears coupled by a chain allows the upper toothed ring (1) to rotate around its axis on the lower toothed ring (2) at specific angles, obtaining the shear between the two toothed rings (1, 2).

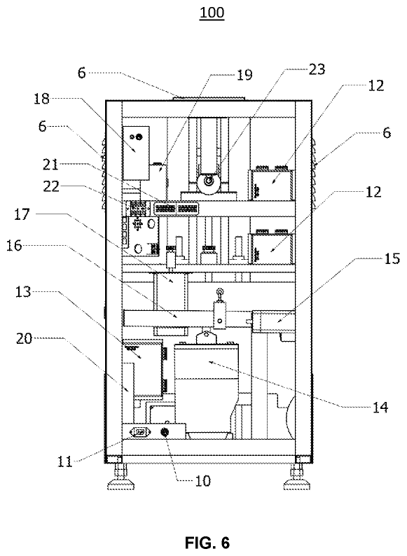

FIGS. 5 and 6 depict the main components that drive a chewing system. An inclined arm or force lever (16) is the main physical component of the in vitro automatic chewing system (100), which allows to apply a force during mastication, which is achieved based on the lever principle, the force will vary according to the distance taken by a lead weight (14) along the inclined arm or force lever (16). According to the force selected on the control panel (3), the lead weight (14) is moved along the inclined arm or force lever (16) by a spindle system which is controlled with a second upper electric motor (15) powered by an electric current source (20) and electronically controlled by the second microstep driver controller (CW-5045) (12) to rotate in both directions. The opposite end of the inclined arm or force lever (16) is connected to an upper shaft (23) that connects to the upper toothed ring (1), as shown in FIG. 5.

FIG. 5 depicts the main components that allow parameter adjustment, control, and automation of the chewing process. A microcontroller board of the Arduino ATMega2560 type (19), electrically powered by a low voltage source (18) is in charge of performing the control and management of the chewing parameters. The standard electronic development board has a series of digital and analog inputs/outputs (21, 22), shown in FIG. 6, which are monitored and controlled using a programming language previously designed specifically for the functions enabled in the equipment. The Arduino ATMega2560 microcontroller card (19) is also in charge of monitoring a series of digital switches for control and protection located in the different mechanisms of the equipment.

According to the described mechanism, a first half of a cycle starts, where the lower toothed ring (2) rises and impacts with the upper toothed ring (1), both together rise to a specific distance, driven by the force of the first lower electric motor (24), at which time the force coming from the inclined arm or the force lever (16) is applied on the solid or semi-solid food positioned on the lower toothed ring (2). At the same time, the second electric motor (17) produces a circular movement at a specific angle of the upper toothed ring (1), obtaining the shear between both toothed rings (1, 2). Once both toothed rings (1, 2) are raised to their highest position, the second half of the cycle begins when the lower toothed ring (2) descends to its lowest position to initiate a new chewing cycle.

### Description of an Operating Method of the Chewing System:

a) Opening the safety cover (5) and placing a solid or semi-solid food sample homogeneously distributed on the lower toothed ring (2).
b) By means of a universal membrane type keypad on the control panel (3), setting the compression force parameters; Once the compression force parameters are set, waiting for the second upper electric motor (15) to move the lead weight (14) through the inclined arm or force lever (16) to a specific position to achieve the preset force that will be used to chew the food.
c) Using the universal membrane type keypad on the control panel (3), setting the parameters for the number of chewing cycles to be used to chew the food.
d) Using the universal membrane type keypad on the control panel (3), setting the parameters for the number of chewing cycles of the shear angle to be used for chewing the food, by means of the inclined arm or the force lever (16).
e) Closing the safety cover (5), and by means of the universal membrane type keypad on the control panel (3), starting the chewing process.
f) Once the chewing process is finished, opening the safety cover (5) and removing the lower toothed ring (2) to extract the masticated sample.

### Example of Application

In a first example of application, to crush a solid or semi-solid food using a specific compression force and a specific shear angle and varying the number of chewing cycles, the safety cover (5) is opened and the food is placed homogeneously distributed on the lower toothed ring (2) and the force, shear angle, and number of chewing cycles that the system will perform are set on the control panel (3). The safety cover (5) is closed and the control panel (3) is started to begin mastication. Once the chewing process is completed, the safety cover (5) is opened and the chewed food located on the lower toothed ring (2) is removed for subsequent size distribution analysis.

In a second application example, to crush a solid or semi-solid food using different compression forces, maintaining the shear angle and the number of chewing cycles, the safety cover (5) is opened and the food is placed homogeneously distributed on the lower toothed ring (2) and the control panel (3) is used to enter the force value, shear angle, and number of mastication cycles that the system will perform. The safety cover (5) is closed and the control panel (3) is started to begin mastication. Once the chewing process is completed, the safety cover (5) is opened and the chewed food located on the lower toothed ring (2) is removed for subsequent size distribution analysis.

1. An in vitro automatic chewing system for solid and semi-solid food (100), CHARACTERIZED in that it comprises:
a lower toothed ring (2) and an upper toothed ring (1) in charge of crushing and generating a shear on a solid or semi-solid food, wherein the lower ring (2) is connected through a connecting rod system to an electric motor (24) that executes in number of chewing cycles controlled by a microcontroller card;
the upper toothed ring (1) is in charge of exerting force and shear on the solid or semi-solid food;
a force lever (16) is connected by a spindle to the upper toothed ring (1);
the force lever is formed by an arm (16) from which a lead weight (14) hangs, which moves along the arm by means of the traction exerted by an electric motor (15) controlled by a microcontroller card; and wherein, the shearing action is provided by an electric motor (17) controlled by a microprocessor card which is connected to the shaft of the upper toothed ring (1) by means of a chain.

2. An in vitro method for automatic chewing of solid and semi-solid foods (100), CHARACTERIZED in that it comprises:
having a lower toothed ring (2) and an upper toothed ring (1) and a safety cover (5), for crushing and generating a shear in a solid or semi-solid food, wherein the lower ring (2) is connected through a connecting rod system to an electric motor (24) which executes in number of chewing cycles controlled by a microcontroller card;
wherein the upper toothed ring (1) is in charge of exerting force and shear on the solid or semi-solid food;
providing a force lever (16) that is connected by a spindle to the upper toothed ring (1);
wherein, the force lever is formed by an arm (16) from which a lead weight (14) hangs, which is moved along the arm by the traction exerted by a second upper electric motor (15) controlled by a microcontroller card; and wherein, the shearing action is provided by an electric motor (17) controlled by a microprocessor card which is connected to the shaft of the upper toothed ring (1) by means of a chain.

3. The automatic chewing method according to claim 1, CHARACTERIZED in that it further comprises:
opening the safety cover (5) and placing a solid or semi-solid food sample homogeneously distributed on the lower toothed ring (2).

4. The automatic chewing method, according to claim 1, CHARACTERIZED in that, by means of a universal membrane type keypad of a control panel (3), parameters of compression force are set; once the compression force parameters are set, waiting for the second upper electric motor (15) to move the lead weight (14) through the inclined arm or force lever (16) to a specific position to achieve the preset force to be used for chewing the food.

5. The automatic chewing method, according to claim 3, CHARACTERIZED in that, by means of the universal membrane type keypad of a control panel (3), the parameters of number of chewing cycles of the shear angle to be used for chewing the food are set, by means of the inclined arm or force lever (16).

6. The automatic chewing method, according to claim 4, CHARACTERIZED in that, in addition, the safety cover (5) is to be closed and by means of the universal membrane type keypad of the control panel (3), and the chewing process is to be started.

7. The automatic mastication method according to claim 4, CHARACTERIZED in that, once the mastication process is completed, the safety cover (5) is opened and the lower toothed ring (2) is removed to extract the masticated sample.
